(19) United States
(12) Patent Application Publication
(10) Pub. No.: US 2010/0204594 A1
(43) Pub. Date: Aug. 12, 2010

(54) MONITORING SYSTEM

(76) Inventor: William Miller, La Jolla, CA (US)

Correspondence Address: Vierra Magen Marcus & DeNiro LLP, 575 Market Street, Suite 2500, San Francisco, CA, 94105, US

(21) Appl. No.: 12/367,116

(22) Filed: Feb. 6, 2009

(51) Int. Cl. A61B 5/04

(52) U.S. Cl. 600/509

(57) ABSTRACT

A monitor comprises a pad having an airtight interior cavity, a pressure transducer, two filtering circuits, a controller, a set of lights and a speaker. An entity sits, lies on, or otherwise applies a force to the pad, which causes a change in pressure within the airtight cavity. The pressure transducer, in communication with the pad, measures the change in pressure and outputs an electrical signal indicative thereof. The filtering circuits filter the output of the transducer to create a signal indicative of breathing of the entity applying force to the pad and a signal indicative of the pulse of the entity applying force to the pad. A controller uses the output of the filters to determine whether the entity is experiencing a breathing condition and/or heart condition. The controller actuates lights, speakers and/or other output devices to report the sensed conditions.

### BACKGROUND

The normal heart will receive electrical signals which cause the ventricles in the heart to compress and eject blood approximately seventy times per minute. One serious abnormality, ventricular fibrillation (hereinafter “VF”), can occur when the electrical signaling system of the heart fails. VF is a rapidly quivering motion of the walls of the ventricles which does not result in the ejection of blood from the ventricle. Sudden cardiac arrest (SCA) and death will usually follow if the VF continues for several minutes. SCA is responsible for 350,000 deaths per year in the U.S. alone.

In some cases, VF has been terminated and the heart returned to normal rhythm by application of an electrical shock supplied by a defibrillator. Devices called Automatic External Defibrillators (AEDs) have been positioned in many public places and are also available for home use. They are designed to be usable by average citizens with very little training. An individual going into VF will quickly lose consciousness and collapse. When this happens in a public place and others are present, the normal procedure is that someone immediately places a call to emergency medical services (e.g., 911) while someone else retrieves an AED and follows the recorded (vocal) instructions provided by the AED.

When paddles from the AED have been attached to the patient, electrical signals picked up by the paddles will be analyzed by the electronics in the AED to determine what the status of the patient is and whether an electrical shock is appropriate or another procedure is to be followed. Many lives have been saved with the help of AED's.

One critical factor in the success of resuscitation efforts using electric shocks is the time elapsed from the start of VF to the application of the shock. It has been estimated that for each minute elapsed the probability of success decreases by ten percent. For VF occurrences in public places, adequate placement of AED's as well as increased public awareness and willingness to assist in these emergency situations should reduce the time elapsed between the onset of VF and defibrillation attempts.

For VF occurrence in private places, or in public places where no observers are present, the existing system does not offer a way to bring about a defibrillation attempt within a few minutes of the onset of VF. Approximately 70% of cardiac arrests take place in the home. AEDs can be bought for use in the home. However, if VF occurs at home, the AED will be of no use unless others in the home are made aware of the occurrence of VF. For example, consider the cases when VF occurs at night and everyone in the house is asleep, or during the day when no one else is in the room where the VF is occurring.

### SUMMARY

The technology described herein pertains to a monitor that can detect the onset of VF and provide an alarm signal to trigger an immediate response to the emergency. Under these circumstances it becomes possible to attempt defibrillation quickly after the onset of VF and, thereby, increase the probability of a successful defibrillation. The monitor described herein can also be used to detect sleep apnea, and other conditions related to pulse and respiration.

One embodiment of a monitor comprises a pad having an airtight interior cavity, a pressure transducer, two filtering circuits, a controller, a set of lights and a speaker. An entity sits, lies on or otherwise applies a force to the pad, which causes a change in pressure within the airtight cavity. The pressure transducer, in communication with the cavity in the pad, measures the change in pressure within the cavity and generates an electrical signal indicative thereof. The filtering circuits filter the output of the transducer to create one signal indicative of breathing of the entity applying force to the pad and another signal indicative of the pulse of the entity applying force to the pad. A controller uses the output of the filters to determine whether the entity is experiencing a breathing condition and/or heart condition. The controller actuates lights, speakers and/or other output devices to report the sensed conditions.

One embodiment of the monitor includes a pad having an airtight interior cavity and a pressure transducer in communication with the pad. The pressure transducer has an electrical output indicative of pressure (including change in pressure) within the cavity in response to an entity applying a force to the pad. The monitor also includes a filtering circuit that receives the electrical output and filters the electrical output to generate a output signal indicative of pulse activity of the entity applying the force. The monitor also includes a controller in communication with the filtering circuit. The controller receives the output signal and tests whether the output signal alternately reaches a first high threshold within a first period of time and a second low threshold within a second period of time. The controller reports a pulse condition for the entity if the output signal does not reach the tested threshold within the appropriate period of time.

One embodiment includes a process that comprises sensing information about pressure within a cavity and generating a first electrical signal indicative of the information about pressure within the cavity, filtering the first electrical signal to generate a second signal indicative of an activity of an entity applying a force to the cavity, determining whether the second electrical signal reaches a high threshold within a first period of time, reporting a condition if it is determined that the second electrical signal does not reach the high threshold within the first period of time, determining whether the second electrical signal reaches a low threshold within a second period of time (after determining whether the second electrical signal reaches the high threshold within the first period of time), and reporting the condition if it is determined that the second electrical signal does not reach the low threshold within the second period of time.

One embodiment includes a process that comprises sensing pressure changing in a cavity in response to an entity applying a force to the cavity and creating a first signal indicative of the pressure change, creating a second signal from the first signal that is indicative of respiration activity of the entity, creating a third signal from the first signal that is indicative if pulse activity of the entity, and determining whether the entity has stopped applying forces to the cavity based on the second signal and the third signal.

### BRIEF DESCRIPTION OF THE DRAWINGS

FIG. 1 is a block diagram of one embodiment of a monitoring device.

FIG. 2 is a sectional view of one embodiment of pad that can be used with the system of FIG. 1.

FIG. 3 is a sectional view of one embodiment of a pressure transducer.

FIG. 4 is sectional view of one embodiment of a pressure transducer.

FIG. 5 is a graph depicting one example of a signal output by a pressure transducer used with the monitor described herein.

FIG. 6 is a schematic diagram of one embodiment of a filter circuit.

FIG. 7 is a graph depicting one example of a signal output by the filter of FIG. 6.

FIG. 8 is a schematic diagram of one embodiment of a filter circuit.

FIG. 9 is a graph depicting one example of a signal output by the filter of FIG. 8.

FIG. 10 is a graph of the signal form FIG. 7 showing two thresholds for testing the data.

FIG. 11 is a graph of the signal form FIG. 9 showing two thresholds for testing the data.

FIG. 12 is a graph depicting one example of a signal output from the filter of FIG. 6 when a user stops applying forces to the pad.

FIG. 13 is a graph depicting one example of a signal output from the filter of FIG. 8 when a user stops applying forces to the pad.

FIG. 14 is a flow chart describing one embodiment of a process for operating the monitor described herein.

FIG. 15 is a flow chart describing one embodiment of a process for performing the pulse data analysis.

FIG. 16 is a flow chart describing one embodiment of a process for performing the respiration data analysis.

FIG. 17 is a flow chart describing one embodiment of a process for performing the off-pad analysis.

FIG. 18 is a flow chart describing one embodiment of a process for reporting alarms.

### DETAILED DESCRIPTION

A monitor is described herein that is non-invasive and will generate an electrical signal characteristic of the heart's response to the normal stimulating electrical signal seen on an electrocardiogram. The monitor can also be used to detect that breathing motion has stopped or has been reduced below an acceptable rate or amplitude. This information is then used to alert about VF, sleep apnea or other conditions.

One embodiment of the monitor comprises a pad having an airtight interior cavity, a pressure transducer, one or more filtering circuits, a controller, one or more lights, and one or more speakers. An entity sits, lies on, or otherwise applies a force to the pad, which causes a change in pressure within the airtight cavity. The pressure transducer, in communication with the airtight cavity, measures the change in pressure and outputs an electrical signal indicative thereof. The filtering circuits filter the output of the transducer to create a signal indicative of breathing of the entity applying force to the pad and a signal indicative of the pulse of the entity applying force to the pad. The controller uses the output of the filters to determine whether the entity is experiencing a breathing condition (e.g., sleep apnea) and/or heart condition (e.g., VF). The controller actuates the lights, speakers or other output devices to report the sensed conditions. Variation of the above-described components can also be used. Additionally, in one embodiment, to prevent the reporting of false alerts, the monitor can detect that the entity is no longer applying forces to the pad (e.g., the entity had gotten off the pad).

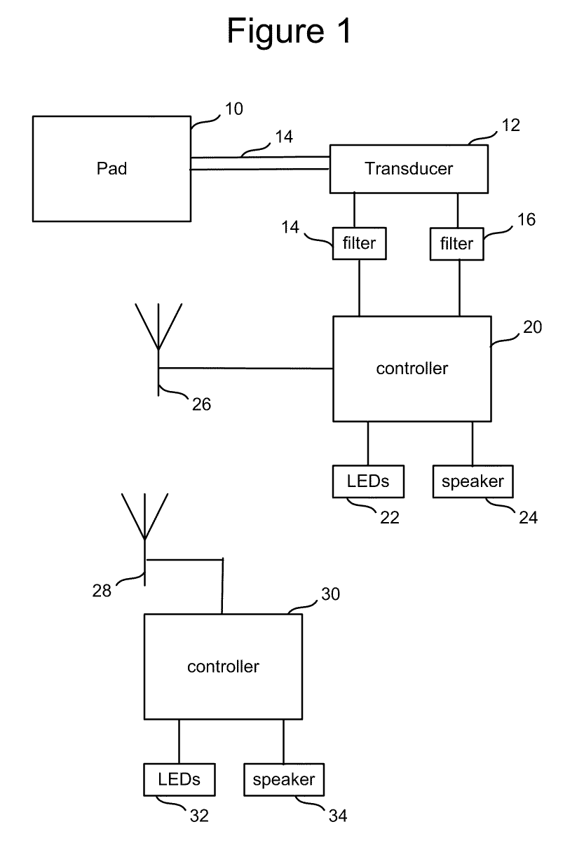

FIG. 1 provides one example of a monitoring system that uses the technology described herein. FIG. 1 shows pad 10 connected to flexible tube 14. At the other end of flexible tube 14 is pressure transducer 12. In one embodiment, pressure transducer 12 fits inside the end of flexible tube 14. In some embodiments, pad 12 or flexible tube 14 will include a valve for connecting to a pump in order to add air and adjust the pressure of pad 10.

In one embodiment, pad 10 is an air-tight flexible enclosure which forms an airtight cavity. The enclosure is made of plastic or other material that can be airtight and is flexible. Many different types of enclosures can be used and the technology described herein is not limited to any one particular type of enclosure. In some embodiment, the enclosure is isolated from the ambient atmosphere so air does not pass between the enclosure and the atmosphere.

In some embodiments, an open-cell foam pad can be inside the enclosure. In other embodiments, the cavity can be empty (other than air). For example, pad 10 can be an air mattress. If no foam pad is present, it is useful to pressurize the air inside the cavity of pad 10 to slightly greater than ambient atmosphere pressure so that pad 10 will hold its shape, and the top and bottom of pad 10 will not touch when a human (or other entity) lies on the pad.

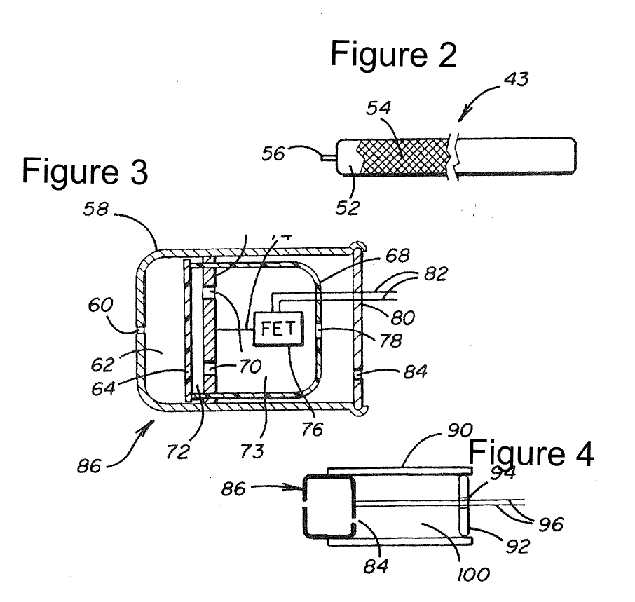

FIG. 2 provides an example of pad 10, implemented as a self-inflating mattress 43. The self-inflating mattress 43 is made by filling the mattress shell 52 with open-celled foam 54 which is bonded to the interior of mattress shell 52. The foam 54 should have a relatively low stiffness and there should be just enough foam to cause the mattress 43 to expand to its full size when there is no load on the mattress.

Pad 10 can be located in many different places. For example, pad 10 can be used in conjunction with a bed. Pad 10 can be placed between the mattress cover and the mattress. Alternatively, pad 10 can be on top of the mattress.

Pad 10 can also be used on a chair. In such a case, pad 10 can be made to match the lateral dimensions of the chair seat so that the user will sit on the pad. The pad can be on top of a chair cushion and/or underneath upholstery that hides pad 10. The pad could also, in principle, be incorporated into clothing or upholstery.

There are no electrical wires going to the pad. The pad is, therefore, completely noninvasive. Flexible (e.g., plastic) tube 14 which connects pad 10 to pressure transducer 12 is an air connection only. There are no electrical wires in tube 14.

Pad 10 will be affected by motion when a person (or other entity) being monitored is either sitting or lying on the pad. The motion of the person (or other entity) will affect the pressure in the airtight cavity of pad 10. The pressure generated in the pad can be understood with reference to Newton's Second Law of Motion: F=ma. In this formula, “F” is the total of all external forces acting on a mass “m”, and “a” is the acceleration of the center of gravity of the mass. If a mass “m” is lying motionless on a pad, then the acceleration of the mass is zero; therefore, the total external force on the mass must be zero. Since gravity is pulling downward on the mass with a force mg, where “g” is the acceleration due to gravity (32 ft/sec².) there must also be an equal and opposite (upward) force, mg, on the body. This force is supplied by pad 10. When the mass “m” is placed on the pad 10, the air in the pad is compressed thereby having its pressure increased, and it is the excess (over atmospheric) pressure in the pad which exerts the upward force on the body. The system described below is designed to ignore the steady state pressure in the pad. The system, however, is interested in motions within the body. As these varying motions occur the center of gravity of the body is accelerating. In order for these accelerations to occur there must be external forces on the body (see previous discussion of F=ma). These forces come from the pad and they are due to excess pressure in the pad. Pressure transducer 12 measures these excess pressures, and thereby provides information about the motion of the entity that is on top of pad 10.

Transducer 12 (see FIG. 1) measures the pressure (including change of pressure) inside the cavity of pad 10 and generates an electrical output indicative of this pressure. That electrical output is provided to filter 14 and filter 16. In one embodiment, filter 14 and filter 16 are separate electrical circuits both receiving the same signal from transducer 12. In other embodiments, filters 14 and 16 can receive different information from transducer 12. Alternatively, filter 14 and filter 16 can be part of the same electrical circuit.

One example of an appropriate transducer is an electret condenser microphone (ECM) 86, shown in FIG. 3. ECMs generally designed for use in the audio range (e.g., 20 to 20 KHz) are commercially available. In order for an ECM to detect the pressure variations within the interior cavity of pad 10, the response of the ECM must be extended to frequencies below 20 Hz.

ECM 86 has a cylindrical aluminum shell 58 having input opening 60 which permits pressure variations to reach air space 62 in front of flexible condenser plate 64. Aluminum shell 58 is crimped around a circular flat circuit board 80. Air space 72, between movable plate 64 and the fixed plate 66, is connected via two holes 70 in the fixed plate to the air space 73 behind the fixed plate in order to prevent motion of the flexible plate from producing a large pressure variation in the region 72. The combined region 72 and 73 is bounded by a rigid plastic shell 68 and flexible capacitor plate 64. Shell 68 has a small hole 78. As the permanently polarized flexible plate (the electret) 64 responds to the pressure changes in the space 62 and causes the distance between the two condenser plates to vary, the voltage between the plates will vary. The voltage between the plates is applied to the input terminal 74 of the field effect transistor (FET) 76. The output leads of the FET 76 are shown at 82. There is some leakage between the air space 62 and the combined spaces 72 and 73. If the pressure in the region of 62 rises by some fixed amount and is maintained at this elevated value, then the plate 64 will initially move toward plate 66. However, as air leaks from region 62 to the regions 72 and 73, the pressure in region 72 will reach the same value as that in region 62, plate 64 will return to its original position and the voltage output to FET 76 will return to zero. The time required for the pressure in regions 62 and 72 to equalize depends on the leakage rate and the volume of the regions 72 and 73. For example, if a steady pressure increment is applied to region 62 and the pressure in region 72 rises to this value in about one-tenth of a second, the response of the ECM will fall off of frequencies below 10 Hz.

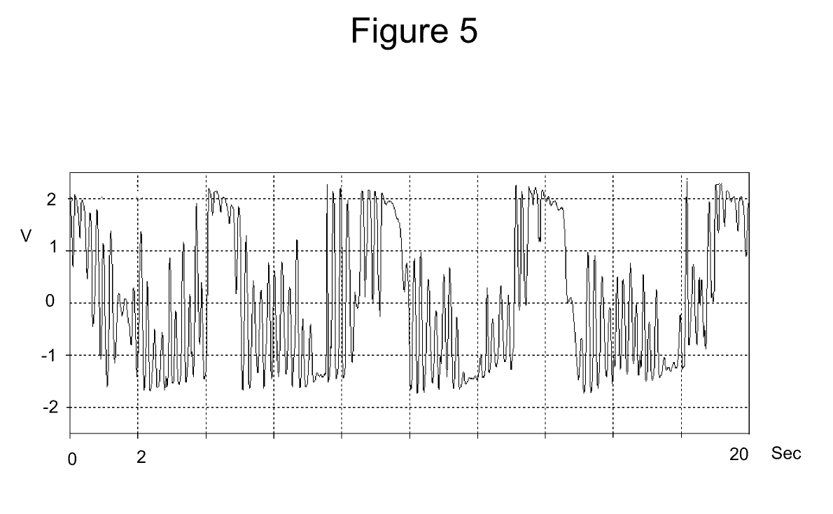

The response of ECM 86 can be extended to lower frequencies by slowing the rate at which the pressure equalizes in regions 62 and 72. This can be done either by slowing the leakage rate or by adding to the volume in regions 72 and 73. The latter method can be accomplished by an external modification to microphone. In FIG. 4, microphone 86 has a cylinder 90 fitted snugly over the microphone 86. A plate 92 seals the far end of cylinder 90. Wires 96 from the field effect transistor 96 exit through small hole 94 and plate 92. An air-tight seal is applied in the hole 94 around wires 96. A small hole 84 has been bored through the circuit board 80 to connect regions 72 and 73 in the microphone with region 100; thereby, increasing the time required for the leakage to cause equalization of the pressures in regions 62 and 72.

Note that other types of pressure sensors/transducers can also be used. The technology described herein is not limited to one particular type of pressure sensor.

Another example of a sensor useful in the monitoring technology described herein is an electric pressure sensor of the type used in digital scales. Sensors of this type provide an electrical signal representative of the pressure exerted on the sensor. An electronic sensor of this type could be placed under one or more legs of a chair or a bed or incorporated into one or more legs of the chair or bed.

The output of pressure transducer 12 is an electrical signal that is indicative of the pressure and/or change of pressure inside the cavity of pad 10. FIG. 5 is a graph (voltage versus time) of one example of an output electrical signal provided by transducer 12. In one embodiment, this signal is provided to both filters 14 and 16 depicted in FIG. 1. The graph in FIG. 5 shows transistor voltage versus time for a 200 pound man sitting on the pad. This voltage is proportional to the pressure variation in the pad. Two of the main features of the trace are a slower repetitive pressure variation with a frequency of approximately 0.3 Hz and a more rapid but damped pressure oscillation with a frequency of about 5 Hz. The slow repetitive pressure variation provides data about respiration. The more rapid damped pressure oscillation provides data about pulse of the body sitting on pad 10.

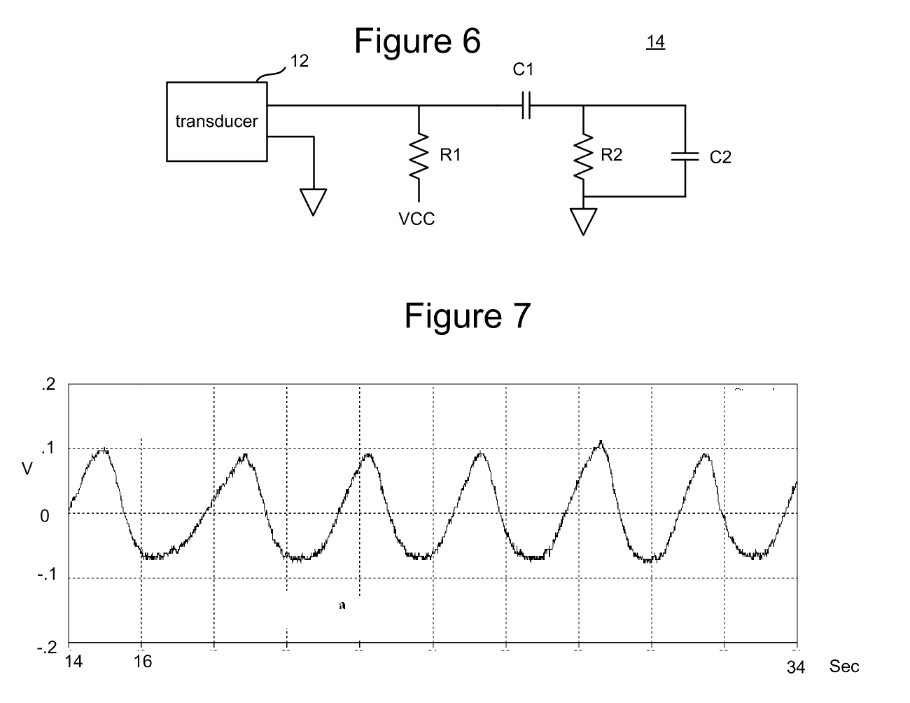

FIG. 6 is a schematic diagram of one embodiment of filter 14. The purpose of filter 14 is to suppress the more rapid variations, thereby, isolating the slower repetitive pressure variations with a frequency of about 0.3 Hz, corresponding to respiration data. FIG. 6 shows transducer 12 providing one terminal to ground and another terminal to resistor R1 and capacitor C1. The opposite side of resistor R1 is connected to VCC (power supply). The other end of capacitor C1 is connected to resistor R2 and capacitor C2. The other ends of resistor R2 and capacitor C2 are connected to ground. In one embodiment, R1 is a 10K ohms resistor, R2 is a 14.6K ohms resistor, C1 is a 680 uF capacitor and C2 is a 220 uF capacitor.

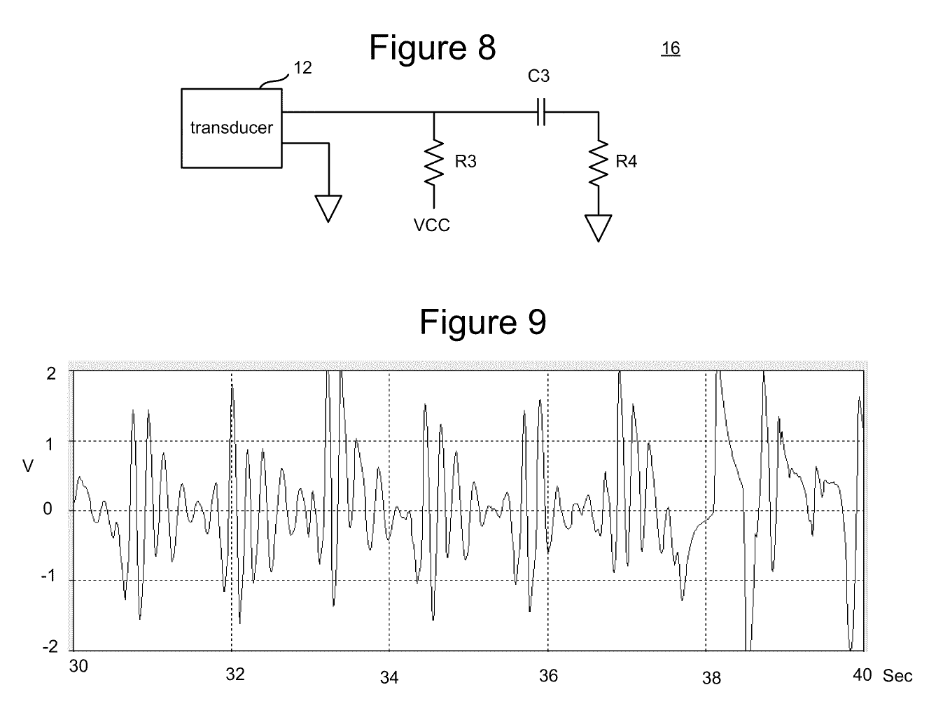

FIG. 7 is a graph depicting voltage versus time for the output of the circuit of filter 14 (FIG. 6). The voltage depicted in FIG. 7 is measured across capacitor C2. This voltage is proportional to the change in pressure inside pad 10 due to a person (or animal) breathing while sitting (or lying) on top of pad 10.

FIG. 8 is a schematic diagram of a filter circuit providing one example of implementation of filter 16. FIG. 8 shows transducer 12 having one terminal connected to ground and a second terminal connected to resistor R3 and capacitor C3. The other end of resistor R3 is connected to VCC. The other end of capacitor C3 is connected to resistor R4. The other end of resistor R4 is connected to ground. In one example, R3 is a 10K ohms resistor, R4 is a 14.7K ohms resistor and C3 is a 4.7 uF capacitor.

FIG. 9 is a graph of voltage versus time which shows the output voltage of filter 16. The voltage depicted in FIG. 9 is measured across resistor R4. This voltage is proportional to the pressure variations in pad 10 due to the heart beating of the person (or animal) sitting on pad 10.

Looking back at FIG. 1, the output voltages from filter 14 and filter 16 are provided to controller 20. Various types of processers can be used to implement controller 20. For example, the PIC12F675 from Microchip Technology Inc. is one example of a suitable controller that can be used to implement controller 20. Another example of a suitable controller is the RFPIC12F675 from Microchip Technology Inc. Other processors can also be used. In one embodiment, controller 20 is programmable, and includes flash memory (or other nonvolatile storage) to store software that programs controller 20 to perform the processes described below. In other embodiments, a specialized processor can also be developed. No particular type of processor is required for controller 20.

Controller 20 is in communication with a set of one or more light-emitting diodes (LEDs) 22 and one or more speakers 24. Based on the data received from the filters, controller 20 will determine which sounds and/or lights to actuate in order to report the current condition(s). If an alarm is to be sounded, controller 20 will activate the appropriate LEDs and the appropriate speakers to indicate the appropriate sounds and sights.

In one embodiment, controller 20 includes an onboard RF transmitter. For example, the RFPIC12F675 controller includes an onboard transmitter. Thus, FIG. 1 shows controller 20 in communication with antenna 26 for communication with a remote reporting device. In one embodiment controller 20 can send an indication of the current condition(s) and/or alarms (via antenna 26 to antenna 28) to a remote display device. FIG. 1 shows a remote display device which includes antenna 28, controller 30, one or more LEDs 32 and one or more speakers 34. The information about the current condition(s) and/or alarms are sent to controller 30 which will light up the appropriate LEDs 32 and cause the appropriate sounds on speakers 34. This way, if the monitor is placed with a person sleeping in a bedroom, the remote display device (controller 30, LED 32 and speaker 34) can be placed elsewhere in the house or building so that someone else can monitor the person sleeping.

Controller 20 makes use of a set of status flags. In one embodiment, the status flags are registers or locations in memory set aside to act as flags. The table below indicates an example set of eight flags; however, other sets of flags can also be used. More details of the flags are provided below.

| FLAG | FUNCTION |
|---|---|
| 0 | Toggles every ½ second |
| 1 | Set for off-pad test enabled |
| 2 | Set for pulse alarm enabled |
| 3 | Set for respiration alarm enabled |
| 4 | Set/clear for high/low test (pulse) |
| 5 | Set/clear for high/low test (respiration) |
| 6 | Set for off-pad alarm enabled |
| 7 | Toggles every 1/10 second |

Controller 20 will analyze the voltage outputs from filters 14 and 16 and determine the status of the body sitting on pad 20. In one embodiment, there are four statuses: Normal status, sleep apnea, VF, off-pad. In another embodiment, there can be a fifth status to report VF with agonal breathing (pulse signal reduced and large gaps between breaths). In the normal status, the patient's pulse is normal and breathing is normal. Thus, the outputs of filters 14 and 16 will be as depicted in FIGS. 7 and 9. Controller 20 generates a sleep apnea alarm when the respiration signal stops for more than a predetermined short time period. Controller 20 generates a VF alarm when the pulse signal is reduced. In some embodiments, controller 20 will determine that the body is in VF if the pulse signal is reduced and respiration stops. An off-pad indication is provided when controller 20 determines that the person being monitored gets off the pad and, therefore, is no longer applying a force to the pad. For example, the person gets out of the bed or gets off of the chair (e.g., stands up).

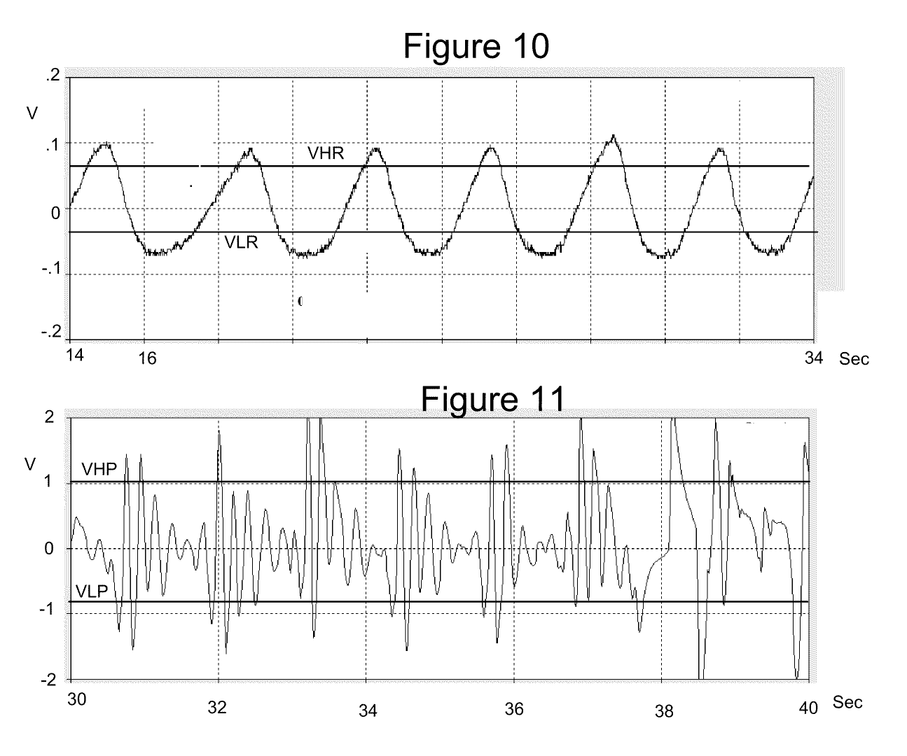

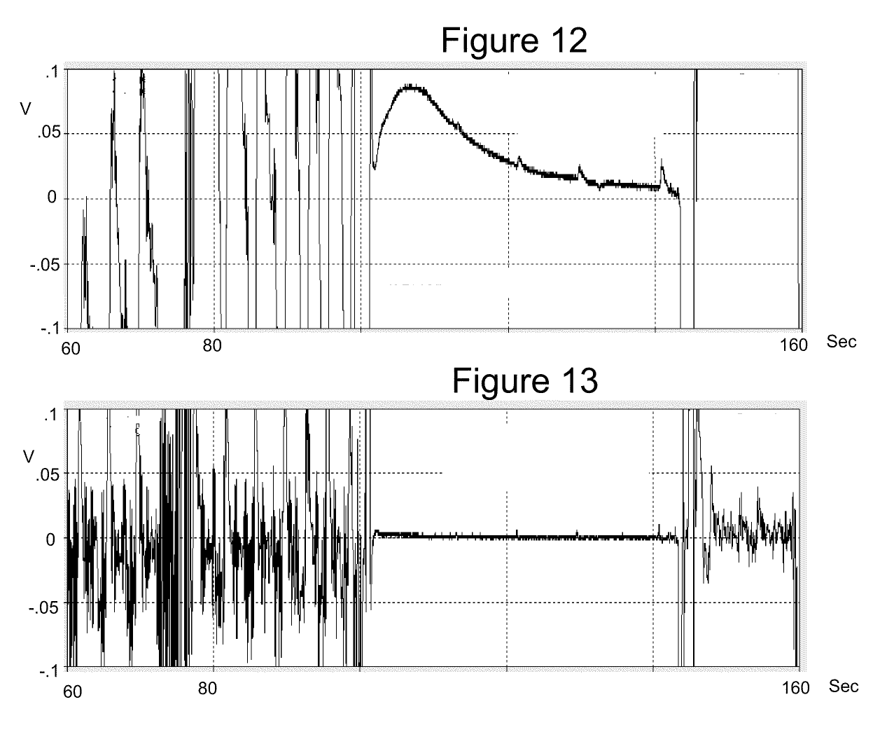

To collect and analyze the data, controller 20 has a data cycle with a duration of 20 milliseconds. The pulse signal (from filter 16, see FIG. 9) and the respiratory signal (from filter 14, FIG. 7) are sampled once in each data cycle, which corresponds to a data rate of 50 cycles per second. The goal of the analysis is to determine whether or not the respiration and pulse signals are oscillating up and down with sufficient amplitude. The analysis can be explained with reference to FIG. 10 and FIG. 11. FIG. 10 corresponds to FIG. 7, and FIG. 11 corresponds to FIG. 9. FIG. 10 includes two thresholds: VHR and VLR. The threshold VHR represents a high voltage threshold for respiration data and the threshold VLR represents a low voltage threshold for respiration data. When each piece of data is read, the system is either doing a high-pass test or a low-pass test. In the high-pass test for respiration data, controller 20 is determining whether the signal from the filter has exceeded VHR. When controller 20 is performing a low-pass test for respiration data, controller 20 will determine whether the signal has become less than VLR. For example, looking at FIG. 10, if the reading is being taken at point A on the trace, and the system is in the high-pass test mode, then the reading obtained is compared with the value of VHR. At point A the voltage will be less than VHR. Thus, the test has failed and the system will increment a counter which keeps track of the time elapsed since the last successful test sand remain in the high-pass test mode until either a test is passed or a sufficient number of failures have occurred. When a high test is passed, as at point B in FIG. 10, the system will switch to a low test mode, and reset the counter. On the other hand, If there are a sufficient number of failures before the test is passed, then controller 20 will conclude that the patient is in sleep apnea and will sound the alarm In one embodiment, the counter is set to overflow (reaches predetermined tripping point) after 10 seconds of consecutive failures.

FIG. 11 shows a high voltage threshold VHP for pulse data and a low voltage threshold VLP for pulse data. When data is sampled, the controller 20 is either doing a high-pass test or a low-pass test. In the high-pass test for pulse, controller 20 is determining whether the signal from filter 16 has exceeded VHP. When controller 20 is performing a low-pass test for pulse data, controller 20 will determine whether the signal has become less than VLP. In one embodiment, each time the test fails a counter will be incremented. When the counter reaches a predetermined value, the alarm will be sounded. Once the test passes, the system will switch to low-pass test mode. For example, around point B, the high-pass test mode will pass and the system will switch into low-pass test mode. In one embodiment, the counter is set to overflow (reaches predetermined tripping point) after 10 seconds of consecutive failures

FIG. 12 is a graph of voltage versus time for the data in the respiration channel which is the output of filter 14. The graph of FIG. 12 shows the data recorded from transducer 12 when a user gets off the pad at time 100 sec. At that point, the data increases and then slowly decays until time 140. During that time the data is above a positive voltage value (dependent upon the particular implementation). (During this period, (i.e., after the user gets off the pad) the data in this channel does not represent respiration, but a redistribution of air in the measurement system)

FIG. 13 is a graph of voltage versus time for the data, in the pulse channel, which is the output of filter 16. The graph of FIG. 13 shows the data recorded from transducer 12 when a user gets off the pad at time 100 sec. At that point, the voltage signal goes to zero volts and remains relatively close to zero volts while the user is off the pad.

In one embodiment, controller 20 will test for the pulse signal dropping to zero and the respiration signal remaining high for a period in excess of 10 seconds. In another embodiment, controller 20 will test for the pulse signal no longer varying by more than a predetermined amount and the respiration signal remaining above a predetermined level for more than a predetermined period of time. When these two conditions are met, the off-pad alarm will be set.

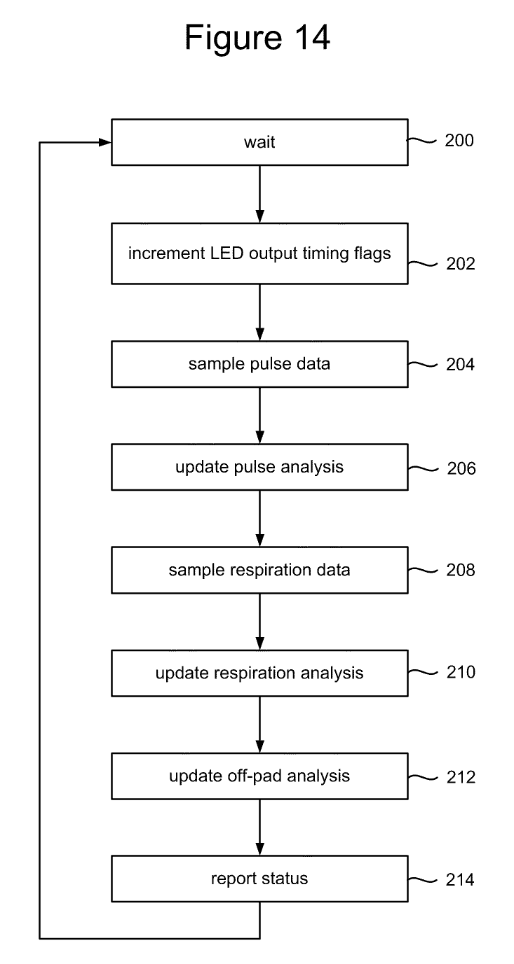

FIG. 14 is a flow chart describing one embodiment of a process for operating the monitor system described herein. In one embodiment, the process of FIG. 4 is performed by controller 20 at the direction of software that programs controller 20. The software can be stored in volatile (e.g., DRAM) or non-volatile memory (e.g., flash memory) within controller 20 or in volatile or non-volatile memory outside of and connected to controller 20. In step 200, controller 20 will wait for the next data cycle. For example, one embodiment will include 50 data cycles per second. In another embodiment, controller 20 will not wait (will not perform step 200), but instead will continuously performs data cycles. Once a data cycle is started, in step 202 controller 20 will increment the LED output timing flags. In one embodiment, flag 0 and flag 7 will be used to cause the LEDs to blink. In order to do this, these flags will be toggled. In one example implementation, flag 7 is made to toggle between clear and set positions 10 times per second, which means it will be set for one-tenth of a second, cleared for one-tenth of a second, set for one-tenth of a second, etc. Thus, every one-tenth of a second (step 202), the flag 7 must be toggled. In a similar manner, flag 0 is made to toggle back and forth two times per second. Therefore, an LED that is to be blinked once per second will follow flag 0 and an LED to be blinked 5 times a second will follow flag 7. Both of these flags are updated in step 202.

In step 204, controller 20 samples and stores pulse data from filter 16. In step 206, controller 20 will update the pulse data analysis, as discussed below. In step 208, controller 20 samples and stores the respiration data from filter 14. In one embodiment, controller 20 will include analog to digital converters. The outputs of the filters will be provided to the analog to digital converters and a digital sample will be obtained in steps 204 and 208. In step 210, the respiration analysis is updated based on the new sampled data, as discussed below. In step 212, controller 20 will perform the off-pad analysis, discussed below. In step 214, controller 20 will report the status of the two analyses. That status can be reported by turning on/off the appropriate LEDs, sending appropriate sound through the speakers, transmitting the alerts to a remote display device, displaying the alerts on a monitor/display, sending an email, sending a text message, sending an Instant Message, sending a page, updating a website, etc. Additionally, all (or a subset of) the alerts will be stored in nonvolatile memory (or volatile memory) for controller 20. In one embodiment, controller 20 will report any one of normal condition, insufficient heart rate (e.g., VF), or insufficient breathing (e.g., sleep apnea). Other conditions can also be reported.

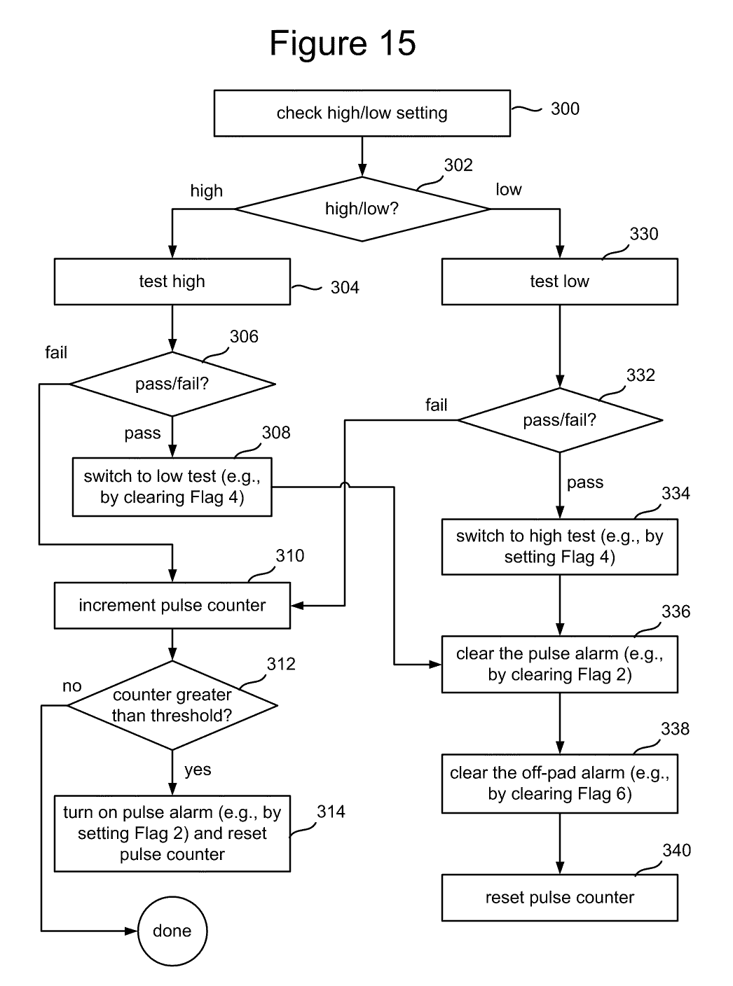

FIG. 15 is a flow chart describing one embodiment for updating the pulse analysis. For example, the process of FIG. 15 is one example implementation of step 206 of FIG. 14. In step 300 of FIG. 15, controller 20 will check the high/low setting stored in flag 4 to determine whether to test for the high threshold or test for the low threshold. If flag 4 indicates a test for the high threshold, then in step 304 the system will test for the high threshold. For example, looking at FIG. 11, the system will determine whether the magnitude of the signal is greater than VHP. If the magnitude of the signal is greater than VHP (step 306) then the test passes and in step 308 controller 20 will switch to the low test by clearing flag 4.

If the most recently sampled magnitude is less than the high threshold (step 306), then the test has failed and a counter (referred to as the pulse counter) will be incremented in step 310. In one embodiment the system will maintain a count of the number of consecutive failures. This counter will be incremented in step 310. In step 312, it is determined whether that counter that was incremented in step 310 is now greater than a threshold. For example, the threshold 500 failures (corresponding to 10 seconds). If the counter is not greater than the threshold, then the process of FIG. 15 is completed. If the counter is greater than the threshold, then the pulse alarm is turned on by controller 20. In one embodiment, the pulse alarm is turned on by setting flag 2 in step 314. Additionally, the pulse counter (that was incremented with step 310) is reset to zero in step 314. In one embodiment, setting the pulse alarm is an indication that the body is experiencing VF.

If the test for the high threshold passes (step 306) because the more recent sample is greater than the VHP, then the system switches to the low test by clearing flag 4 in step 308 and clearing the pulse alarm in step 336. One embodiment includes clearing flag 2 in step 336. In step 338, the off-pad alarm is also cleared. For example, flag 6 can be cleared. In step 340, the pulse counter is reset to zero.

If, in step 302, it is determined that the system is in the low pass test mode, then in step 330 the system will test to determine whether the magnitude of the voltage sampled from the filter is below the low threshold. For example, controller 20 will determine whether the voltage of FIG. 11 is below VLP. If the test fails because the magnitude of the voltage sampled from the filter is not below the low threshold, then the process continues at step 310. If the low pass test passes because the magnitude of the voltage sampled from the filter is below the low threshold, then at step 334 the system will switch to the high pass test. For example, flag 4 can be set. After setting flag 4, the process continues at step 336. In this manner, the pulse alarm will be set when a sufficient number of fails happen in a row without an intervening pass.

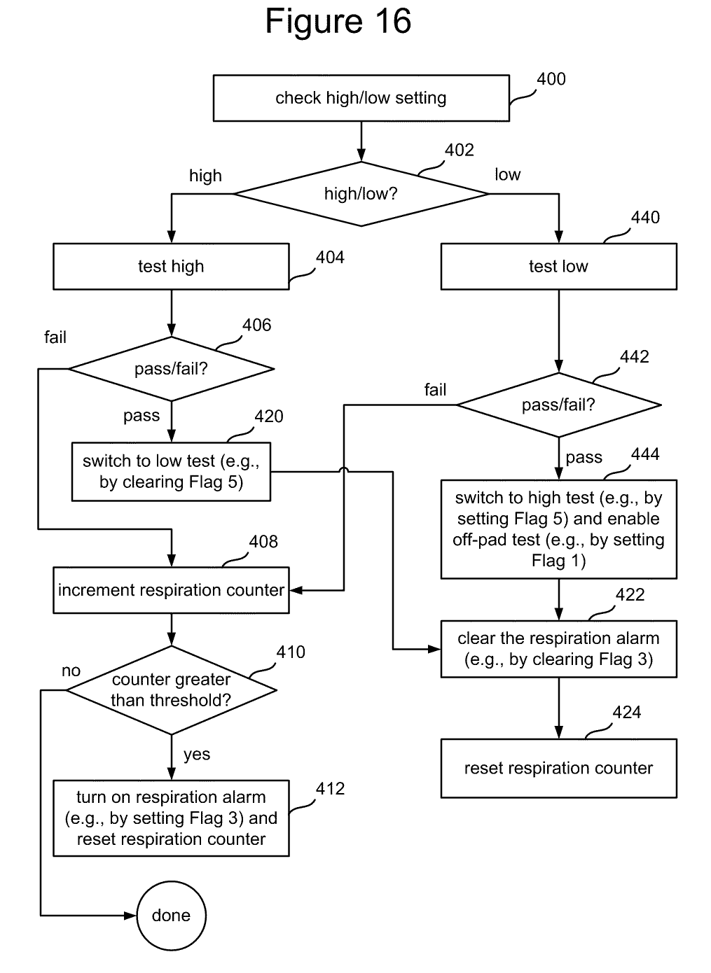

FIG. 16 is a flow chart describing one embodiment of a process for updating the respiration analysis. For example, the process of FIG. 16 can be used to implement step 210 of FIG. 14. In step 400 of FIG. 16, controller 20 will check the high/low setting. For example, controller 20 will check flag 5 to determine whether the controller should check for the high pass threshold or the low pass threshold. If the flag indicates to check for the high pass threshold (step 402) then the system will test for the high pass threshold of step 404. For example, controller will test the respiration data to determine whether the magnitude of the data is greater than VHR. If the magnitude of the current sample of data is not greater than VHR (step 406), then in step 408 a counter (referred to as the respiration counter) is incremented. Similar to the pulse data, a count is kept of the number of consecutive failures. Whenever the consecutive failures reaches the threshold (tested for in step 410), the system will report the respiration alarm. In one embodiment, the respiration counter is set to indicate an alarm after 10 seconds of consecutive failures. In one implementation, testing for ten seconds includes counting to 500. In step 410, controller 20 determines whether the counter (incremented in step 408) is greater than the threshold. If not, the process of FIG. 16 is complete. If the counter is greater than the threshold, then controller 20 sets the respiration alarm. In one embodiment, setting the respiration alarm is performed by setting flag 3 and resetting the respiration counter in step 412. In one embodiment, setting the respiration alarm is an indication that the body is experiencing sleep apnea.

If it was determined in step 406 that the test passed, then in step 420 controller 20 will switch to the low pass test. For example, controller 20 can clear flag 5. In step 422 the respiration alarm will be cleared. For example, flag 3 can be cleared. If the flag is already cleared, then there will be no change. In step 424, the respiration counter will be reset.

If, in step 402, controller 20 determines that it was in low pass test mode, then in step 440 a low pass test is performed. For example, controller 20 will determine whether the magnitude of the currently sampled respiration data is below VLR. If the magnitude of the data is not below VLR then the test fails, the process will continue at step 408 and the respiration counter will be incremented. If the data was below VLR, and the test passed controller 20 will switch to the high pass test. In one embodiment, setting the respiration alarm is an indication that the body is experiencing sleep apnea. For example, flag 5 can be set. In addition, step 444 includes enabling the off-pad test to be performed later. In one embodiment, the off-pad test is enabled by setting flag 1. When the off-pad test is not enabled, the system will not test for an off-pad status.

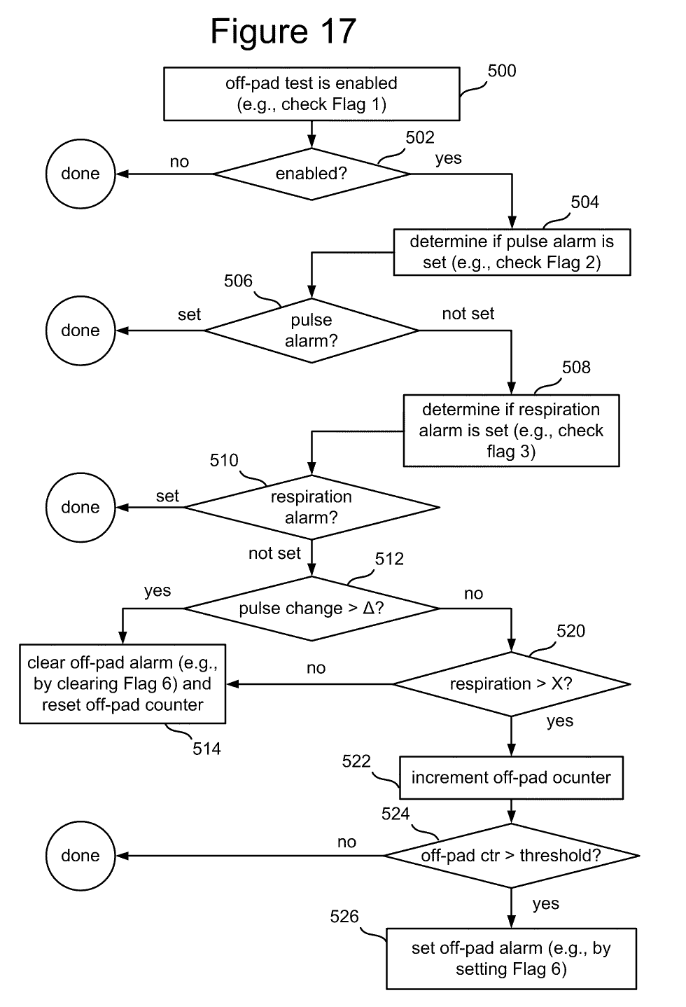

FIG. 17 is a flow chart describing one embodiment of a process for performing the off-pad analysis. For example, the process of FIG. 17 is one example of an implementation of step 212 of FIG. 14. In step 500, controller 20 determines whether the off-pad test is enabled. For example, controller 20 can check flag 1 to see whether the off-pad test is enabled (see step 444, FIG. 16). If the off-pad test is not enabled (step 502) then the process of FIG. 17 is complete and controller 20 will not perform the off-pad test. If the off-pad test is enabled (step 502), then in step 504 controller 20 will determine if the pulse alarm is set. In one example, controller 20 will check to see if flag 2 is set. If the pulse alarm is set (see step 506) then there is no need to perform the off-pad analysis because the controller knows the patient is in VF and not off-pad. If the pulse alarm is not set (see step 506) then in step 508 controller 20 determines whether the respiration alarm is set. For example, controller 20 can check flag 3. If the respiration alarm is set (see step 510) then the process of FIG. 17 is completed because there is no need to check for an off-pad condition since controller knows the user is in sleep apnea. If the respiration alarm is not set, then in step 512, controller 20 will determine whether the pulse data has changed, during a time period of 6 sec (or another suitable period), by more than an amount Δ. In one embodiment, Δ is set at one-tenth of a volt which is one fiftieth of the full range of the Analog to Digital Converter. If the pulse data has changed by Δ during the relevant time period, then the system clears the off-pad alarm (if it is set) by clearing flag 6 and resets the off-pad counter in step 514. If the pulse data has not changed by Δ during the relevant time period, then the process continues at step 520.

Note that another characteristic of the OFF PAD condition is evident in FIG. 12, where the pressure in the respiration channel remains well above zero for several seconds. The system will test (in step 520) whether the respiration data is greater than some predetermined magnitude X. In one embodiment, X can be one fiftieth of the full range of the A/D converter above the midpoint of the Y-coordinates. If the data in the respiration channel is not greater than X, then the process continues at step 514 because it is assumed that there is no off-pad condition. If the respiration data is greater than X then an off-pad counter is incremented in step 522. Controller 20 will keep a counter for counting the number of times the respiration data is greater than X while the pulse data is not changing by more than Δ. In step 524, it is determined whether the off-pad counter is greater than a threshold. If not, the process of FIG. 17 is complete for this particular data cycle. If the off-pad counter is greater than a threshold which in one embodiment is a counter value corresponding to six seconds, then in step 526, controller 20 will set the off-pad alarm. In one embodiment, the off-pad alarm is set by setting flag 6.

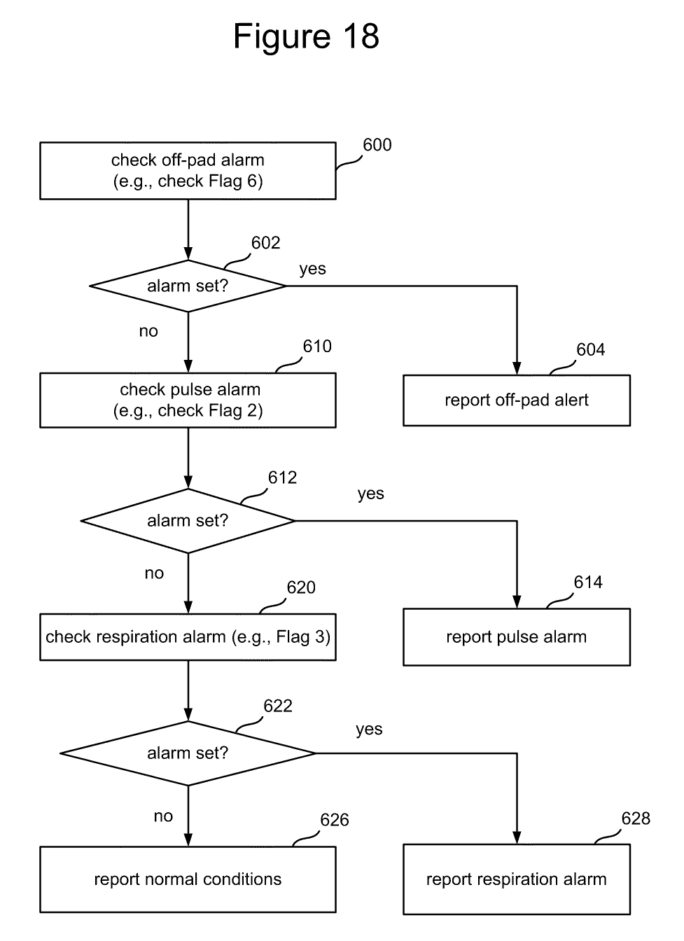

FIG. 18 is a flow chart describing one embodiment of a process for reporting the status determined by controller 20. For example, the process of FIG. 18 can be one example of an implementation of step 214 of FIG. 14. In step 600 of FIG. 18, controller 20 will check the off-pad alarm. For example, controller 20 can check flag 6. If the off-pad alarm is set (step 602), then controller 20 will report an off-pad alert in step 604. If the off-pad alarm is not set (step 602), then in step 610 controller 20 will check the pulse alarm. For example, checking the pulse alarm can include check whether flag 2 is set. If the pulse alarm is set (step 612), then in step 614 controller 20 will report a pulse alarm. For example, the pulse alarm could indicate that the body is in a VF condition. If the pulse alarm was not set (step 612), then in step 620 controller 20 checks whether the respiration alarm has been set. One example of step 620 is checking whether flag 3 is set. If the respiration alarm is not set, then a normal condition will be reported by controller 20 in step 626. If the respiration alarm is set, then controller 20 will report a respiration alarm at step 628. The respiration alarm is one example of reporting that the body is experiencing sleep apnea.

There are various ways for reporting the alarm conditions as well as normal conditions. In one embodiment, reporting normal conditions in step 626 includes causing the speaker to be silent and causing the green light from the LEDs to be solid. Alternatively, a particular tone can be sounded for normal conditions and different tones can be sounded for different alarms. Other means for reporting normal conditions can also be used. In one embodiment, reporting a pulse alarm in step 614 can include toggling a red LED off and on five times a second and providing audio alert on the speaker. In another embodiment, either reporting the respiration alarm of step 628 (or reporting the off-pad alarm at step 604) can be performed by blinking the red light on and off once a second and using a different audio alarm (or no audio alarm). In another embodiment, different color LEDs can be used to report the off-pad alarm, pulse alarm and respiration alarm. No particular method for turning the lights on and off or providing audio alert is required for the technology described herein. For any of the alarms, the LEDSs can be used to indicate a condition without using the speaker. For example, a sleep apnea alarm may include a blinking LED, but no speaker sound.

Additionally, the system can store data in response to an alarm. For example, if a respiration (e.g., sleep apnea) condition or pulse condition occurs, the Controller (in steps 614 or 628) can write the respiration data, pulse data and any analysis to a file which can be stored in memory (or a re-writeable disk or flash memory drive) local to and connected to the Controller. In one embodiment, the system would include a USB port to connect a portable flash memory drive. The Controller (in steps 614 or 628) can write the respiration data, pulse data and any analysis to one or more files on the flash memory drive which can be removed by a health care provider, who can then read the files and determine whether and how to treat the patient.

Other methods can be used to analyze the data being generated in the pulse and respiration channels described above. For example, the autocorrelation function for the series of pressure values in the two channels can be calculated. Repetitive patterns in the signals and the characteristic times between repetitions can then be determined.

Another approach would be to do Fourier analysis of pressure values in the two channels, in order to identify the major frequency components of the signals. These, and other signal processing techniques, require a more powerful microprocessor chip which differs somewhat from the controller chip described above.

The foregoing detailed description of the invention has been presented for purposes of illustration and description. It is not intended to be exhaustive or to limit the invention to the precise form disclosed. Many modifications and variations are possible in light of the above teaching. The described embodiments were chosen in order to best explain the principles of the invention and its practical application, to thereby enable others skilled in the art to best utilize the invention in various embodiments and with various modifications as are suited to the particular use contemplated. It is intended that the scope of the invention be defined by the claims appended hereto.

What is claimed is:

1. A monitor, comprising:
an enclosure having an interior cavity;
a pressure transducer in communication with the enclosure, the pressure transducer having an electrical output indicative of pressure within the cavity in response to an entity applying a force to the enclosure;
a filtering circuit receiving the electrical output and filtering the electrical output to generate an output signal indicative of pulse activity of the entity applying the force; and
a controller in communication with the filtering circuit and receiving the output signal, the controller tests whether the output signal alternately reaches a first high threshold within a first period of time and a first low threshold within a second period of time, the controller reports a pulse condition for the entity if the output signal does not reach the appropriate tested first high threshold or first low threshold within the appropriate period of time.

2. The monitor of claim 1, wherein:
the filtering circuit includes a first filter and a second filter;
the first filter generates the output signal indicative of the pulse activity; and
the second filter filters the electrical output to generate an electrical signal indicative of respiration activity of the entity.

3. The monitor of claim 2, wherein:
the controller tests whether the electrical signal indicative of respiration activity alternately reaches a second high threshold within a third period of time and second low threshold within a fourth period of time, the controller reports a breathing condition for the entity if the electrical signal indicative of respiration activity does not reach the appropriate tested second high threshold or second low threshold within the appropriate period of time.

4. The monitor of claim 2, wherein:
the controller determines whether the entity stops applying the force to the enclosure based on the output signal indicative of pulse activity of the entity and the electrical signal indicative of respiration activity of the entity.

5. The monitor of claim 2, wherein:
the controller determines that the output signal indicative of pulse activity of the entity is at a constant value and that the electrical signal indicative of respiration activity of the entity remains above a minimum value for some period of time; and
the controller reports that the entity has stopped applying any force to the enclosure in response to determining that the output signal indicative of pulse activity of the entity is at the constant value and that the electrical signal indicative of respiration activity of the entity remains above the minimum value for some period of time.

6. The monitor of claim 1, wherein:
the enclosure comprises an open-cell foam pad within an airtight flexible enclosure; and
the interior cavity is airtight.

7. The monitor of claim 1, further comprising:
a flexible tube connecting the cavity to the pressure transducer, the pressure transducer is located within a portion of the flexible tube, the interior cavity is isolated from ambient atmosphere.

8. The monitor of claim 1, further comprising:
a light in communication with the controller, the controller reports the pulse condition by illuminating the light.

9. The monitor of claim 1, further comprising:
a speaker in communication with the controller, the controller reports the pulse condition by sending an audio signal to the speaker.

10. The monitor of claim 1, wherein:
the filtering circuit includes a first filter and a second filter;
the first filter generates the output signal indicative of the pulse activity;
the second filter filters the electrical output to generate an electrical signal indicative of respiration activity of the entity;
the controller tests whether the electrical signal indicative of respiration activity alternately reaches a second high threshold within a third period of time and second low threshold within a fourth period of time, the controller reports a breathing condition for the entity if the electrical signal indicative of respiration activity does not reach the appropriate tested threshold within the appropriate period of time;
the controller determines whether the entity stops applying the force to the enclosure based on the output signal indicative of pulse activity of the entity and the electrical signal indicative of respiration activity of the entity;
the enclosure comprises an open-cell foam pad within an airtight flexible enclosure; and
the monitor further comprises an output device in communication with the controller for reporting the breathing condition, the pulse condition and whether the entity has stopped applying any force to the enclosure.

11. The monitor of claim 1, wherein:
the first period of time is equal to the second period of time.

12. The monitor of claim 1, further comprising:
a wireless transmitter;
a receiver remote from and in wireless communication with the wireless transmitter, the wireless transmitter receives the reporting of the pulse condition and transmits the reporting to the remote receiver; and
an output device in communication with the remote receiver, the output device is actuated by the receiver to alert of the pulse condition.

13. A method for monitoring, comprising:
sensing information about pressure within a cavity and generating a first electrical signal indicative of the information about pressure in the cavity;
filtering the first electrical signal to generate a second electrical signal indicative of an activity of an entity applying a force to the cavity;
determining whether the second electrical signal reaches a high threshold within a first period of time;
reporting a condition if it is determined that the second electrical signal does not reach the high threshold within the first period of time;
determining whether the second electrical signal reaches a low threshold within a second period of time, after the determining whether the second electrical signal reaches the high threshold within the first period of time; and
reporting the condition if it is determined that the second electrical signal does not reach the low threshold within the second period of time.

14. The method of claim 13, wherein:
the activity is a breathing activity and the condition is lack of sufficient breathing.

15. The method of claim 13, wherein:
the activity is a pulse activity and the condition is lack of sufficient pulse.

16. The method of claim 13, further comprising:
filtering the first electrical signal to generate a third electrical signal indicative of breathing of the entity;
determining whether the third electrical signal reaches a high value within a third period of time;
reporting a breathing problem if it is determined that the third electrical signal does not reach the high value within the third period of time;
determining whether the third electrical signal reaches a low value within a fourth period of time, after the determining whether the third electrical signal reaches the high value within the third period of time; and
reporting the breathing problem if it is determined that the third electrical signal does not reach the low value within the second period of time.

17. The method of claim 16, further comprising:
determining that the entity is no longer applying any forces to the cavity based on the second electrical signal and the third electrical signal.

18. The method of claim 16, further comprising:
determining that the second electrical signal is at a constant value and that the third electrical signal remains above a minimum value for some period of time; and
reporting that the entity has stopped applying any force to the pad in response to determining that the second electrical is at the constant value and that the third electrical signal remains above the minimum value for some period of time.

19. The method of claim 13, wherein:
the reporting the condition includes actuating a light.

20. The method of claim 13, wherein:
the steps of determining whether the second electrical signal reaches the high threshold and determining whether the second electrical signal reaches the low threshold are repeated periodically.

21. A method for monitoring, comprising:
sensing pressure changing in a cavity in response to an entity applying a force to the cavity and creating a first signal indicative of the pressure change;
creating a second signal from the first signal that is indicative of respiration activity of the entity;
creating a third signal from the first signal that is indicative if pulse activity of the entity; and
determining whether the entity has stopped applying forces to the cavity based on the second signal and the third signal.

22. The method according to claim 21, further comprising:
automatically determining whether the third signal does not meet a threshold within a period of time; and
reporting that the entity has a pulse condition in response to determining that the third signal does not meet a threshold within a period of time.

23. The method according to claim 22, further comprising:
automatically determining whether the second signal does not meet a threshold within a period of time; and
reporting that the entity has a breathing condition in response to determining that the second signal does not meet a threshold within a period of time.

24. The method according to claim 21, further comprising:
reporting that the entity has stopped applying forces to the cavity.

25. The method according to claim 21, wherein determining whether the entity has stopped applying forces to the cavity comprises:
determining whether the second signal remains above a particular level and determining whether the third signal does not change by a predetermined amount.

26. A heart activity monitor for a human, comprising:
an enclosure having an interior cavity;
a pressure transducer in communication with the interior cavity of the enclosure, the pressure transducer creating an first electrical signal indicative of changes in pressure within the cavity in response to motion of the human;
an electrical detector responsive to the first electrical signal, the electrical detector generating a second electrical signal indicative of pulse activity of the human; and
a controller responsive to the second electrical signal, the controller determining whether the output signal reaches a first high threshold within a first period of time and a first low threshold within a second period of time, and reporting a pulse condition for the human if the output signal does not reach the first high threshold or the first low threshold within the respective first and second periods of time.

27. A monitor, comprising:
an enclosure having an interior cavity;
a pressure transducer in communication with the enclosure, the pressure transducer having an electrical output indicative of pressure within the cavity in response to an entity applying a force to the enclosure;
a filtering circuit receiving the electrical output and filtering the electrical output to generate an output signal indicative of breathing activity of the entity applying the force; and
a controller in communication with the filtering circuit and receiving the output signal, the controller tests whether the output signal alternately reaches a first high threshold within a first period of time and a first low threshold within a second period of time, the controller reports a pulse condition for the entity if the output signal does not reach the appropriate tested first high threshold or first low threshold within the appropriate period of time.
